(19) United States
(12) Patent Application Publication
(10) Pub. No.: US 2015/0282815 A1
(43) Pub. Date: Oct. 8, 2015

(54) BONE CHISEL AND METHOD FOR WORKING A TIBIA HEAD

(71) Applicant: IMPLANTCAST GMBH, Buxtehude (DE)

(72) Inventors: Georg Gosheger, Munster (DE); Jens Sass, Buxtehude (DE)

(73) Assignee: IMPLANTCAST GMBH, Buxtehude (DE)

(21) Appl. No.: 14/439,724

(22) PCT Filed: Nov. 8, 2013
(86) PCT No.: PCT/EP2013/003368

§ 371 Date: Apr. 30, 2015

(30) Foreign Application Priority Data
Nov. 9, 2012 (DE) 10 2012 021 901.6

(51) Int. Cl. A61B 17/16

(52) U.S. Cl. CPC A61B 17/1604

(57) ABSTRACT

A bone chisel for creating a protrusion bearing cruciate ligament attachments from an upper side of a tibia head, which has a blade that encloses an area of a projection in a U-shape to a front with a transverse wall and to a side with side walls, and which has a cutter. A guide rod extended parallel to a driving direction of the bone chisel is mounted on a side of the bone chisel formed by the transverse wall, which guide rod is displaceably mounted in a direction of the guide rod in a guide head, which can be affixed on a front side of the tibia head.

### BACKGROUND OF INVENTION

1. Field of Invention

The invention relates to a bone chisel, and a method for working a tibia head.

2. Brief Description of Related Art

The tibia head is the upper thickened end of the human shin bone. It forms the lower part of the knee joint, the upper part of which joint is comprised of the lower end of the femur, which lower end bears two condyles which rest on the tibia head (and on the menisci disposed between the tibia head and the lower end of the femur).

When the knee joint suffers severe injury, a knee endoprosthesis is employed which in its customary form has a tibia plate on its side facing the tibia, which tibia plate is fixed to the tibia head, for which purpose part of the tibia head is excised, e.g. by means of a bone saw, leaving a smooth flat surface.

When this relatively simple surgery is performed, care must be taken to avoid tearing the cruciate ligaments which extend from the middle of the upper side of the tibia head. This would result in their undesirable removal. In the absence of the cruciate ligaments, the patient would subsequently experience disadvantageous weakness of the knee, and disadvantageous sensory deficiencies due to absence of important proprioceptors in the cruciate ligaments.

A knee endoprosthesis which preserves the cruciate ligaments is described in US 2011/0190898 A1. To prepare for application of the tibia plate, the region around the tibia head surface bearing the cruciate ligament connections is excised, leaving this region in the form of a projection (protrusion) on the upper side of the tibia head, the remainder of which tibia head is now removed. A tibia plate is employed which has a U-shaped recess to accommodate the described projection.

The working of the upper part of the tibia head to produce this projection is attended by appreciable risks. Parts of the tibia head immediately adjoining the projection which one desires to leave undisturbed are removed, e.g. by operations of milling, chiseling, sawing, or the like. A small error may suffice to injure the projection which bears the cruciate ligaments, and to injure the cruciate ligaments themselves.

The underlying problem of the present invention was to provide the surgeon with means of reducing these risks.

This problem is solved with the bone chisel and method for working a tibia head as disclosed herein.

### BRIEF SUMMARY OF THE INVENTION

According to the invention, a bone chisel is provided which is used in the excision to prepare the projection. The chisel has a thin U-shaped blade member which is applied so as to generally surround the projection, whereby it (its cutting edge) can be driven into the tibia head, in the longitudinal direction of the tibia. This results in stamping-out of a projection which exactly matches the tibia plate which will later be applied. It further facilitates excision of the material around the projection, with minimal risk, and in particular without injury to the projection or to the cruciate ligaments, which are protected by the blade member. Because the U-shaped blade member is open on one side, it may be applied with this opening directed posteriorly. Thus it can be applied between the tibia head and the femur, in a manner such that it generally surrounds the cruciate ligaments, whereby the cruciate ligaments are undamaged during the entire operation. The manner in which the bone chisel is guided during the driving process ensures the exact proper configuration of the result, with a simple manner of functioning, and in particular provides optimal protection of the cruciate ligaments.

The U-shaped blade member may be in the form of a rounded U shape, but advantageously it may have right angles. With such a configuration of the blade member, the tibia plate may also have a right-angled recess.

The inventive bone chisel may be struck with a hammer on its rear thin edge. However, advantageously the blade member may be formed on a solid shaft body which may have appreciable mass and which provides a suitable impact surface. The shaft body may also serve to facilitate guiding of the blade member with the user's other hand.

Advantageously, the shaft body may be attached to a flange which is formed on the edge of the blade member which is opposite to the cutting edge.

Advantageously, the shaft body has a bent configuration. This allows the positioning of the impact surface of the bone chisel in a region which is readily accessible to a hammer.

Advantageously, the flange may have a sloped configuration, to facilitate its insertion into the narrow (indeed narrowed) region between the femur and the tibia.

A method for working a tibia head using an inventive bone chisel is set forth below.

### BRIEF DESCRIPTION OF THE INVENTION

The invention is illustrated schematically in the drawings, by way of example.

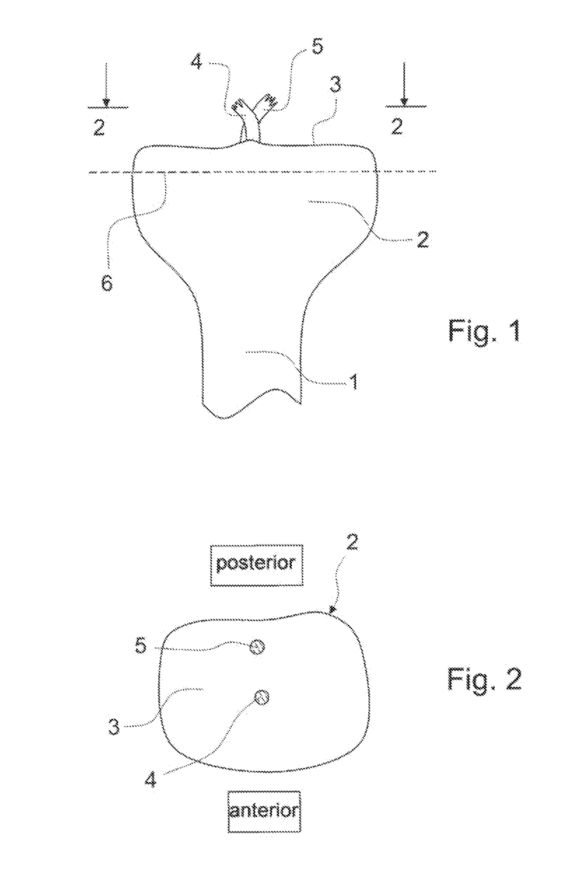

FIG. 1 is an anterior view of an un-worked tibia head;

FIG. 2 is a cross section through line 2-2 of FIG. 1;

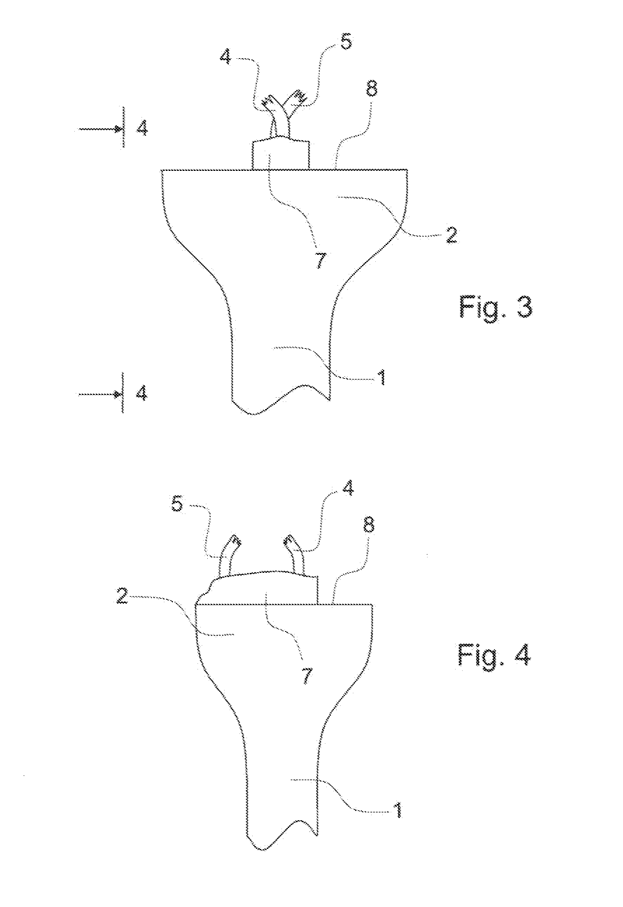

FIG. 3 is a view corresponding to FIG. 1, of a tibia head which has been worked in the area of the cruciate ligaments;

FIG. 4 is a cross section through line 4-4 of FIG. 3;

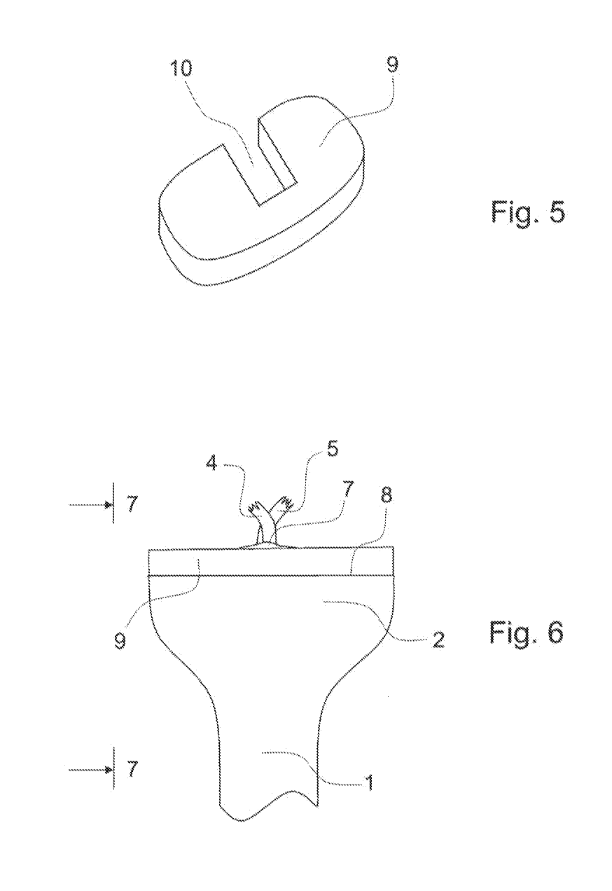

FIG. 5 is a perspective view of a tibia plate;

FIG. 6 is a view corresponding to FIG. 3, with a tibia plate applied;

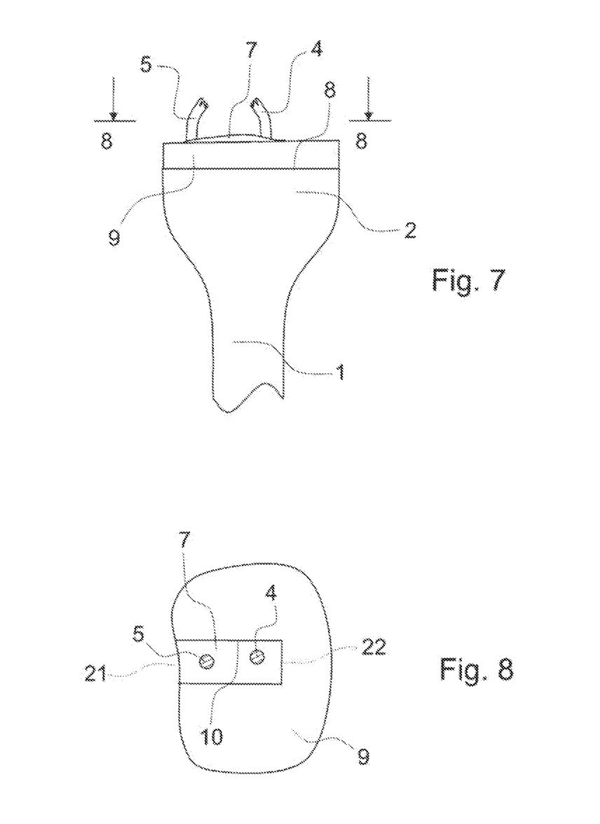

FIG. 7 is a cross section through line 7-7 of FIG. 6;

FIG. 8 is a cross section through line 8-8 of FIG. 7;

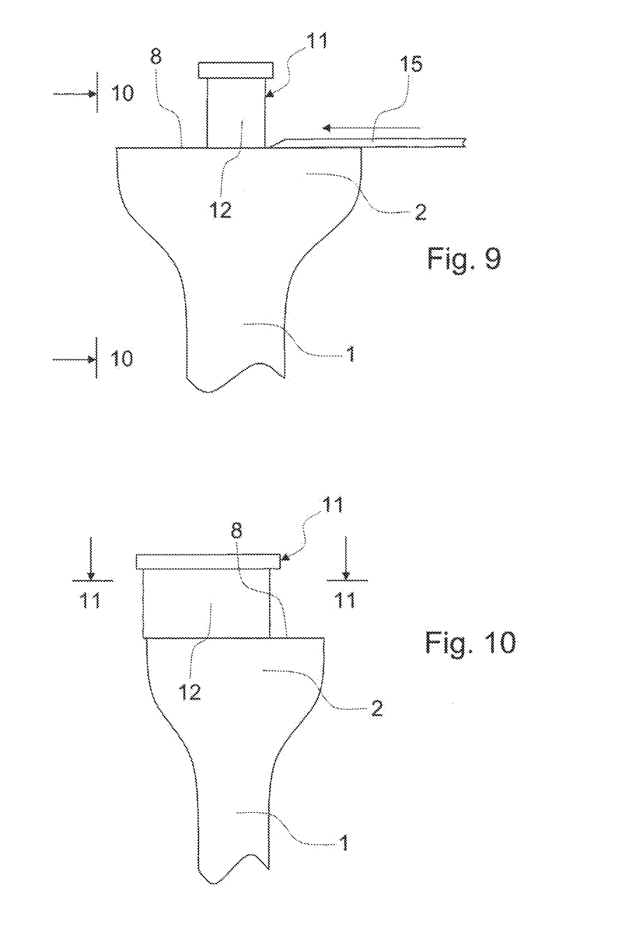

FIG. 9 is a view corresponding to FIG. 3, with the bone chisel inserted;

FIG. 10 is a cross section through line 10-10 of FIG. 9;

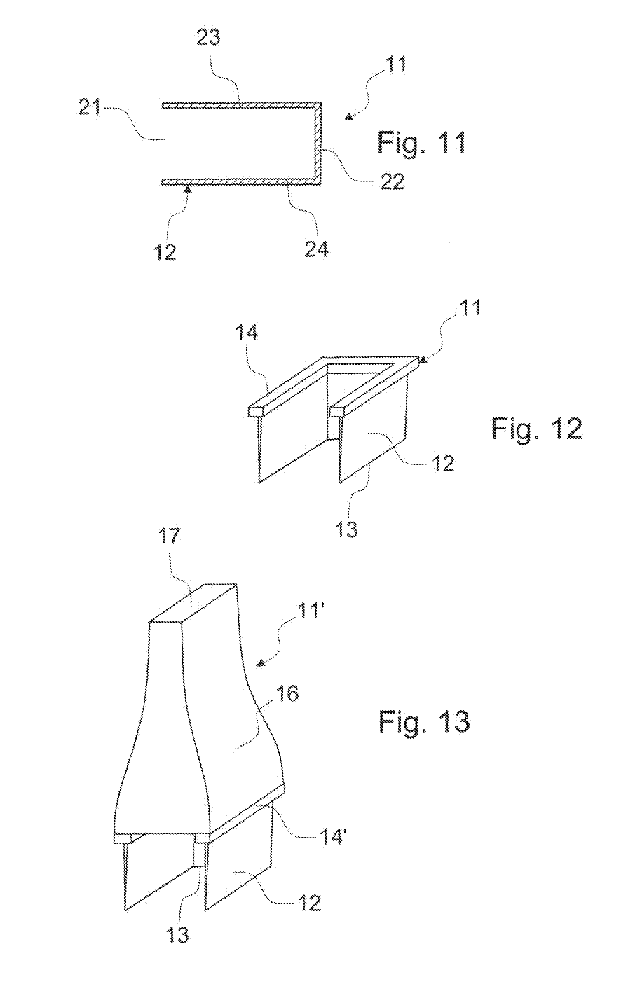

FIG. 11 is a cross section through line 11-11 of FIG. 10, showing a cross section of the bone chisel;

FIG. 12 is a perspective view of the bone chisel illustrated in FIG. 11;

FIG. 13 is a perspective view of a different embodiment of a bone chisel;

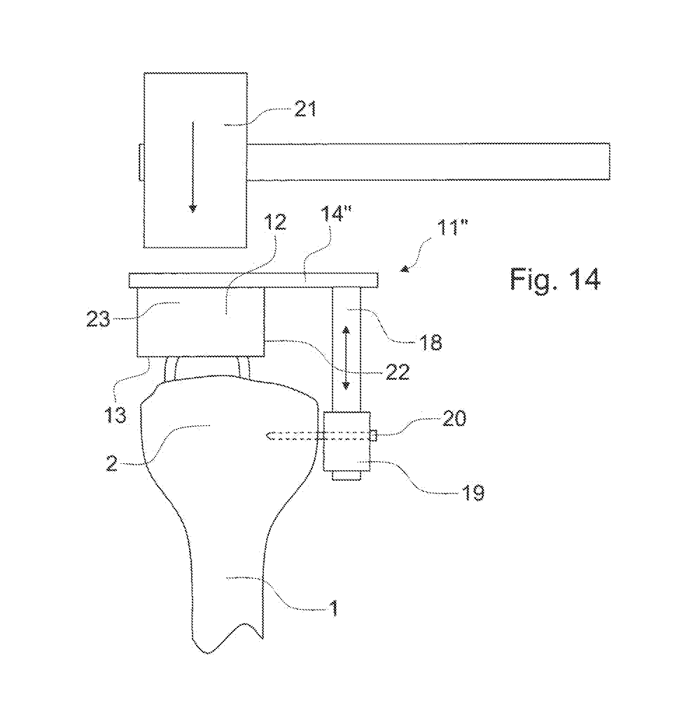

FIG. 14 is a lateral view of a bone chisel provided with longitudinal guide means; and

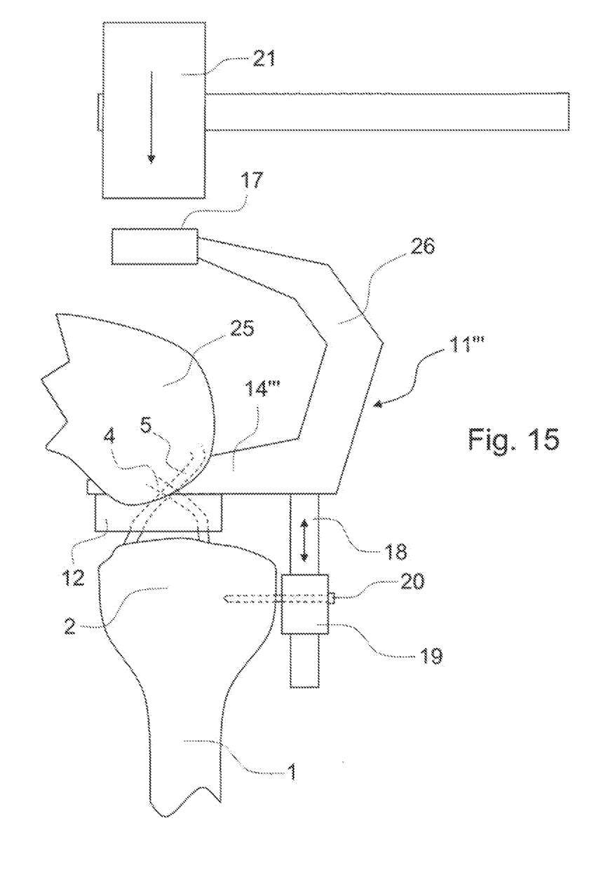

FIG. 15 is a lateral view of a variant embodiment from that illustrated in FIG. 14.

### DETAILED DESCRIPTION OF THE INVENTION

FIG. 1 is an anterior view of the upper region of the tibia 1, thus the shin bone of a man, with the tibia head 2 adjoining the tibia 1 at the top end of the latter. The anterior cruciate ligament 4 and the posterior cruciate ligament 5 are disposed on the upper side 3 of the tibia head 2 (also illustrated in FIG. 2 which is a top view of the upper side 3).

For the sake of clarity, in FIG. 2 the generally used position designations “anterior” and “posterior” are indicated, surrounded by borders.

In the surgical method described in the patent cited earlier in the Specification, for installing a knee endoprosthesis, the tibia head 2 must be worked in the manner shown in FIGS. 3 and 4. The region of the tibia head 2 lying above the dashed line 6 in FIG. 1 is excised, e.g. with a saw. In the process, as illustrated in FIGS. 3 and 4, a projection (protrusion) 7, bearing the cruciate ligaments 4 and 5, is not excised. The cut surface 8 disposed around the projection 7 should be as flat as possible. To achieve this, it is necessary to employ sharp tools around the projection 7, e.g. milling cutters, saws, chisels, or the like.

FIG. 5 illustrates a tibia plate 9 suitable for this surgical method, having a U-shaped recess 10. The periphery of the tibia plate 9 corresponds to the periphery of the cut surface 8 as appears from FIG. 2. The recess 10 corresponds to the periphery of the projection 7. Accordingly, the tibia plate 9 fits on the tibia head 2 which has been excised according to FIG. 3, and can be attached to the cut surface 8 as illustrated in FIG. 6. The attachment may be achieved, e.g., by cementing. The bottom side of the tibia plate 9 may also bear projections (not shown) which may be driven into the tibia head 2 for purposes of attachment. Screws or the like may also be employed in achieving the attachment.

FIGS. 7 and 8 illustrate the arrangement shown in FIG. 6, in a lateral view and a top view. FIG. 8 shows how the recess 10 of the tibia plate 9 fits around the projection 7.

In the process of producing the projection 7 and providing a cut surface 8 which is as flat as possible, sharp tools are employed in the immediate vicinity of the projection 7 and the cruciate ligaments 4 and 5. With such tools, there is a possibility that damage can be caused to the projection 7 and even to the cruciate ligaments 4 and 5. Therefore, according to the present invention, a bone chisel as illustrated in a first embodiment in FIGS. 9 to 12 (bone chisel 11) is employed.

The bone chisel 11 has a peripheral U-shaped blade member 12 which in this embodiment of the bone chisel 11 has a right-angle configuration, as may be seen in particular from FIGS. 11 and 12. FIG. 12 shows that the blade member is thin and comprises a sharp cutting edge 13 which extends around the U shape. A U-shaped peripheral thickened flange 14 is disposed at the upper edge above the cutting edge 13, which flange provides better load-bearing characteristics when the upper edge of the blade member 12 is struck by a hammer. This flange 14 serves also for stabilizing the U shape, but it is possible to omit it.

FIG. 11, which is a cross sectional view through line 11-11 in FIG. 10, shows that the bone chisel 11 illustrated in FIGS. 9-15 generally surrounds a U-shaped region, wherewith its blade member 12 is comprised of a transverse wall 22 and two parallel side walls 23 and 24, and has an opening 21.

In the use of the bone chisel 11, the chisel is applied from above with its U-shaped cutting edge 13 being applied against the upper side 3 of the tibia head 2, wherewith it is positioned and oriented such that it is aligned in correspondence with the edge of the cut surface 10 illustrated in FIG. 8. In FIG. 8 the reference lines 21 and 22 are shown as dashed lines for purposes of illustration.

The bone chisel is now driven in with a hammer, until, as illustrated in FIGS. 9 and 10, its cutting edge 13 is at a height corresponding to the (future) cut surface 8. The blade member 12 now surrounds the projection 7, whereby the walls 22, 23, and 24 of the blade member 12 protect the sides of projection 7 which are at substantial hazard (FIGS. 9 and 10). Now means such as the tool 15 (FIG. 9) may now be used to remove all of the material located around the bone chisel 11 which has been driven into the tibia head 2, and above the intended cut surface 8, wherewith the tool 15 may also be employed, e.g., to smooth off the cut surface 8. If by accident during this process the sharp cutting edge of the tool 15 approaches the projection 7, it cannot proceed into the projection 7, because the latter is protected by the blade member 12.

FIG. 13 illustrates a second embodiment of a bone chisel 11′. In the region of the cutting edge 13, the blade member 12, and the flange 14′, the bone chisel 11′ completely corresponds with the above-described bone chisel 11. However, a massive shaft body 16 is disposed on the flange 14′, having an impact surface 17 which can be struck by a hammer, e.g. the hammer 21 illustrated in FIG. 14.

FIG. 14 illustrates a third embodiment of a bone chisel 11″. Here again the cutting edge 13 and blade member 12 are identical to the corresponding elements of bone chisels 11 and 11′. However here the flange 14″ is elongated in the anterior direction, thus beyond the transverse wall 22, and on its elongation it bears a guide rod 18 which is guided in a guiding head 19 in the direction of the double arrow, namely the longitudinal direction of the tibia 1, so as to be movable longitudinally in said direction. The guiding head is fixed to the tibia head 2 by means of the illustrated screws 20. If a hammer 21 is caused to strike the flange 14″, it will drive the bone chisel 11″ from above into the tibia head 2. In the process, the direction and exact positioning of the application of the bone chisel will be ensured by the guiding of the guide rod 18 in the guiding head 19. The guiding head 19 has been fixed to the tibia head 2 in advance, so as to be precisely oriented.

In the Figures the embodiments of the bone chisel 11 have a blade member 12 and a cutting edge 13 with a U-shaped configuration with right angle corners. However, the U-shape may be a rounded U-shape (not shown).

FIG. 15 illustrates the knee shown in FIG. 14, including the associated femur 25 to which the cruciate ligaments 4 and 5 are fixed. In a manner typical of surgeries of this type, the upper leg with the femur 25 is raised as far as possible and is oriented at an angle, so that the blade member 12 of a bone chisel 11′″ can be inserted between the femur 25 and the tibia head 2, as illustrated in FIG. 15.

With this configuration, the blade member 12 surrounds the cruciate ligaments 4 and 5 in a correct protective disposition. However, the normal impact path of the hammer 21 is blocked by the femur 25, which cannot be shifted laterally any farther without tearing the cruciate ligaments 4 and 5.

Therefore, the impact surface 17 of the bone chisel 11′″ in this embodiment is connected to the blade member 12 via a bent piece 26 the bent region of which extends anteriorly around the relevant region of the femur 25, and transmits impact forces from the impact surface 17 to the blade member 12.

The flange 14′″ of the bent piece 26, corresponding to the flange piece 14″ of FIG. 14, is configured so as to be progressively thicker with progression from the opening 21 to the transverse wall 22 of the blade member 12. This improves the stability, while reducing the thickness in the region of the opening 21, while at that location still providing sufficient play (free space) above the flange 14′″ with respect to the femur 25. The femur 25 has two condyles on its knee-side end region which allow passage of the parts 12 and 14′″ of the chisel 11″ without causing damage.

At this point, a method for working a tibia head 2 with the bone chisel 11″ according to FIG. 15 will be described.

First, the knee is exposed without disturbance of the cruciate ligaments 4 and 5, and the femur 25 is inclined maximally with respect to the tibia 1, until the position illustrated in FIG. 15 is achieved.

Then the bone chisel 11″ with its U-shaped blade member 12 and at least the end region of the flange 14″ which adjoins the opening 21 is inserted, from the anterior side, between the tibia head 2 and the femur 25. The blade member 12 is now brought into a position in which it can protect the cruciate ligaments 4 and 5 on all sides, as it surrounds the cruciate ligaments 4 and 5, wherewith the transverse wall 22 of the blade member is directed anteriorly.

Then the bone chisel 11″ with its U-shaped blade member 12 is pounded into the tibia head 2 and is thereby fixed to the tibia head. At this point, the distal end region of the tibia head 2 can be excised outside the walls 22, 23, and 24 of the blade member 12, using a cutting tool 15 as per FIG. 9.

1. A bone chisel for facilitating excisions for producing a projection bearing connections of cruciate ligaments on an upper side of a tibia head, said chisel having a U-shaped blade member which surrounds a region of the projection on an anterior side by a transverse wall and laterally by side walls, wherein said blade member has a cutting edge, wherein a guide rod extending parallel to a driving direction of the bone chisel is mounted on a side of the bone chisel formed by the transverse wall, wherein the guide rod is displaceably mounted so as to be displaceable in a direction of the guide rod, in a guide head, which is affixed on an anterior side of the tibia head.

2. The bone chisel according to claim 1, wherein the blade member is configured with right-angle bends.

3. The bone chisel according to claim 1, wherein the blade member is formed on a shaft body, which bears an impact surface.

4. The bone chisel according to claim 3, wherein the shaft body is disposed on a U-shaped flange which is attached to an edge of the blade member that is opposite to the cutting edge.

5. The bone chisel according to claim 4, wherein the shaft body is in the form of a bent piece.

6. The bone chisel according to claim 4, wherein the flange becomes thicker with progression from an opening to the transverse wall of the blade member.

7. A method of working a tibia head in a knee between a femur and a tibia, the method comprising:
providing a bone chisel according to claim 1;
exposing the knee without disturbance of cruciate ligaments, with the femur inclined maximally with respect to the tibia;
inserting the bone chisel with its U-shaped blade member, from an anterior side, between the tibia head and the femur, into a position in which the blade member generally surrounds the cruciate ligaments and the transverse wall of the blade member is directed anteriorly;
pounding said blade member into the tibia head to excise a distal end region of the tibia head with the aid of the cutting edge outside the walls of the blade member.
